(19) United States
(12) Patent Application Publication
(10) Pub. No.: US 2024/0226076 A1
(43) Pub. Date: Jul. 11, 2024

(54) Inhibitors of Tryptases and Their Use in Treating Allergies

(71) Applicant: The Board of Trustees of the Leland Stanford Junior University, Stanford, CA (US)

(72) Inventors: Vinit MAHAJAN, Los Altos, CA (US); Young Joo SUN, Redwood City, CA (US); Soo Hyeon LEE, Sunnyvale, CA (US); Gabriel VELEZ, Palo Alto, CA (US)

(21) Appl. No.: 18/558,842

(22) PCT Filed: May 9, 2022
(86) PCT No.: PCT/US2022/028333

§ 371 Date: Nov. 3, 2023

Related U.S. Application Data
(60) Provisional application No. 63/186,591, filed on May 10, 2021.

(51) Int. Cl. A61K 31/4418; A61K 45/06; A61P 37/08

(52) U.S. Cl. CPC A61K 31/4418; A61K 45/06; A61P 37/08

(57) ABSTRACT

Methods are provided for treating allergies, asthma, or mast cell activation syndrome with a tryptase inhibitor. In particular, methods of treating allergies, asthma, or mast cell activation syndrome with the tryptase inhibitor, Avoralstat, are disclosed.

### CROSS-REFERENCE TO RELATED APPLICATION

This application claims benefit under 35 U.S.C. § 119(e) of provisional application 63/186,591, filed May 10, 2021, which application is hereby incorporated by reference in its entirety.

### BACKGROUND

Tryptase is a part of the S1-peptidase (trypsin-like protease) family, and there are 5 subtypes of tryptases, including tryptase-alpha, -beta, -gamma, -delta, and -epsilon. Tryptases are secreted by activated mast cells; thus, they have been used as markers for mast cell activation. Mast cells are part of the immune system that is responsible for both innate and adaptive immune responses. However, when mast cells release too many of their granules containing tryptases, it results in severe allergic responses, referred to as mast cell activation syndrome (MCAS). This syndrome is responsible for severe allergic reactions triggered by heat, strong odors, food, or such.

### SUMMARY

Methods are provided for treating allergies, asthma, or mast cell activation syndrome with a tryptase inhibitor. In particular, methods of treating allergies, asthma, or mast cell activation syndrome with the tryptase inhibitor, Avoralstat, are disclosed.

In one aspect, a composition comprising a tryptase inhibitor for use in a method of treating an allergy, asthma, or mast cell activation syndrome is provided.

In certain embodiments, the tryptase inhibitor is Avoralstat.

In certain embodiments, the composition further comprises a pharmaceutically acceptable excipient.

In another aspect, a method of treating a subject for an allergy, asthma, or mast cell activation syndrome is provided, the method comprising administering a therapeutically effective amount of a tryptase inhibitor to the subject.

In certain embodiments, the tryptase inhibitor is Avoralstat.

In certain embodiments, the tryptase inhibitor is administered according to a daily dosing regimen or intermittently.

In certain embodiments, the tryptase inhibitor is administered orally or intravenously.

In certain embodiments, the method further comprises administering an antihistamine, a non-steroidal anti-inflammatory drug, a beta 2-adrenoceptor agonist, or a corticosteroid.

In another aspect, a method of inhibiting tryptase b2 or tryptase g1 is provided, the method comprising contacting tryptase b2 or tryptase g1 with Avoralstat.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following FIGURES.

FIGS. 1A-1E: Structure-based phylogenetic analysis and biochemical inhibition assay identifies Avoralstat as a potential repurposing candidate for Tryptase inhibition. (FIG. 1A) Sequence-based phylogenetic tree of 75 representative peptidase domains with the main clusters highlighted in different colors. Branches are labeled according to the corresponding structures and PDB IDs. (FIG. 1B) Structure-based phylogenetic tree of mammalian S1-peptidase domain structures. Evolutionary distance was inferred using the UPGMA method. Branch lengths correspond to the Cα RMSD (Å) of the pairwise-structural-alignments calculated in 3DPhyloFold. The proteases with the highest structural similarity to Tryptases were KLKB1 and Factor XI. Docking structure and docking score of KLKB1/Avoralstat (FIG. 1C) and Tryptase-b2/Avoralstat (FIG. 1D). (FIG. 1E) Avoralstat selectivity against 70 proteases.

### DETAILED DESCRIPTION OF EMBODIMENTS

Methods are provided for treating allergies, asthma, or mast cell activation syndrome with a tryptase inhibitor. In particular, methods of treating allergies, asthma, or mast cell activation syndrome with the tryptase inhibitor, Avoralstat, are disclosed.

Before the present methods of treating allergies and mast cell activation syndrome with tryptase inhibitors such as Avoralstat are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “an inhibitor” includes a plurality of such inhibitors and reference to “the enzyme” includes reference to one or more enzymes and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “allergy” is meant to encompass any allergic reaction resulting in activation of mast cells or allergen-induced inflammation, such as caused by any ingested or inhaled allergen, occupational allergen, environmental allergen, drug, or any other substance that triggers a harmful immune reaction resulting in activation of mast cells. The term includes severe allergic reactions triggered by heat, strong odors, or food.

The term “treatment” as used herein refers to either (1) the prevention of an allergy, asthma, or mast cell activation syndrome (prophylaxis), or (2) the reduction or elimination of symptoms of allergy, asthma, or mast cell activation syndrome (therapy).

By “therapeutically effective dose or amount” of a tryptase inhibitor (e.g., Avoralstat) or a derivative thereof is intended an amount that, when administered, as described herein, brings about a positive therapeutic response, such as improved recovery from an allergy, asthma, or mast cell activation syndrome. For example, a therapeutically effective dose or amount may prevent or lessen the severity of allergic symptoms such as, but not limited to, hives, angioedema, itchiness, dermatographism, lightheadedness, dizziness, presyncope, syncope, arrhythmia, tachycardia, congestion, coughing, wheezing, nausea, vomiting, and diarrhea; prevent anaphylactic or near-anaphylactic reactions, and/or prevent asthma or mast cell activation syndrome. In addition, a therapeutically effective dose may inhibit tryptases and tryptase-mediated allergic responses. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Substantially purified” generally refers to isolation of a component such as a substance (compound, drug, inhibitor, metabolite, nucleic acid, polynucleotide, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography, gel filtration, and sedimentation according to density.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

The terms “individual”, “subject”, and “patient”, are used interchangeably herein and refer to any vertebrate subject for whom diagnosis, treatment, or therapy is desired, particularly humans. By “vertebrate subject” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

It will be apparent to one of ordinary skill in the art that various changes and modifications can be made without departing from the spirit or scope of the invention.

### Pharmaceutical Compositions

Pharmaceutical compositions comprising a tryptase inhibitor or a derivative thereof can be used to treat an allergy, asthma, or mast cell activation syndrome. The tryptase inhibitor, or a derivative thereof, can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition can also include an antimicrobial agent for preventing or deterring microbial growth. Nonlimiting examples of antimicrobial agents include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the tryptase inhibitor, or a derivative thereof, or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the tryptase inhibitor or a derivative thereof (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A. H., Handbook of Pharmaceutical Excipients, 3rd Edition, American Pharmaceutical Association, Washington, D.C., 2000.

The compositions encompass all types of formulations and, in particular, those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising a tryptase inhibitor or a derivative thereof are in unit dosage form, meaning an amount of a composition appropriate for a single dose, in a premeasured or pre-packaged form.

The compositions herein may optionally include one or more additional agents. Compounded preparations may include a tryptase inhibitor, or a derivative thereof, and one or more other agents for treating an allergy, asthma, or mast cell activation syndrome, such as, but not limited to, mast cell stabilizers, including cromolyn sodium and quercetin; antihistamines, including cetirizine, ketotifen, fexofenadine, loratadine, ranitidine, famotidine, levocetirizine, and diphenhydramine; non-steroidal anti-inflammatory drugs, including aspirin, diflunisal, ibuprofen, dexibuprofen, naproxen, fenoprofen, ketoprofen, dexketoprofen, indomethacin, tolmetin, sulindac, etodolac, ketorolac, piroxicam, meloxicam, tenoxicam, droxicam, mefenamic acid, and meclofenamic acid; beta 2-adrenoceptor agonists, including salbutamol (albuterol); corticosteroids, including budesonide, fluticasone, mometasone and ciclesonide; and the like.

Alternatively, such agents can be contained in a separate composition from the composition comprising the tryptase inhibitor, or the derivative thereof, and co-administered concurrently, before, or after the composition comprising the tryptase inhibitor or the derivative thereof.

### Administration of a Tryptase Inhibitor

At least one therapeutically effective cycle of treatment with a composition comprising a tryptase inhibitor, as described herein, will be administered to a subject for treatment of an allergy, asthma, or mast cell activation syndrome. By “therapeutically effective dose or amount” of a tryptase inhibitor (e.g., Avoralstat) or a derivative thereof is intended an amount that, when administered, as described herein, brings about a positive therapeutic response, such as improved recovery from an allergy, asthma, or mast cell activation syndrome. For example, a therapeutically effective dose or amount may prevent or lessen the severity of allergic symptoms such as, but not limited to, hives, angioedema, itchiness, dermatographism, lightheadedness, dizziness, presyncope, syncope, arrhythmia, tachycardia, congestion, coughing, wheezing, nausea, vomiting, and diarrhea; prevent anaphylactic or near-anaphylactic reactions, and/or prevent asthma or mast cell activation syndrome. In addition, a therapeutically effective dose may inhibit tryptases and tryptase-mediated allergic responses. The exact amount required will vary from subject to subject, depending on the species, age, and general condition of the subject, the severity of the condition being treated, the particular type of tryptase inhibitor administered, or other drugs employed in combination, the mode of administration, and the like. An appropriate “effective” amount in any individual case may be determined by one of ordinary skill in the art using routine experimentation, based upon the information provided herein.

In certain embodiments, multiple therapeutically effective doses of compositions comprising a tryptase inhibitor, and/or one or more other therapeutic agents, such as one or more other agents for treating an allergy, asthma, or mast cell activation syndrome, such as, but not limited to, mast cell stabilizers, including cromolyn sodium and quercetin; antihistamines, including cetirizine, ketotifen, fexofenadine, loratadine, ranitidine, famotidine, levocetirizine, and diphenhydramine; non-steroidal anti-inflammatory drugs, including aspirin, diflunisal, ibuprofen, dexibuprofen, naproxen, fenoprofen, ketoprofen, dexketoprofen, indomethacin, tolmetin, sulindac, etodolac, ketorolac, piroxicam, meloxicam, tenoxicam, droxicam, mefenamic acid, and meclofenamic acid; beta 2-adrenoceptor agonists, including salbutamol (albuterol); corticosteroids, including budesonide, fluticasone, mometasone and ciclesonide; or other medications will be administered. The compositions comprising a tryptase inhibitor are typically, although not necessarily, administered orally, via injection (subcutaneously, intravenously, or intramuscularly), by infusion, topically, or locally. Additional modes of administration are also contemplated, such as intraocular, intra-arterial, intravascular, pulmonary, intralesional, and so forth.

The preparations according to the invention are also suitable for local treatment. For example, compositions comprising a tryptase inhibitor may be administered locally to the site of allergic inflammation. The particular preparation and appropriate method of administration can be chosen to target the tryptase inhibitor to sites of mast cell activation. Local treatment may avoid some side effects of systemic therapy.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising a tryptase inhibitor, or a derivative thereof, and/or other agents may be administered using the same or different routes of administration in accordance with any medically acceptable method known in the art.

In another embodiment, the pharmaceutical compositions comprising a tryptase inhibitor and/or other agents are administered prophylactically, e.g., to prevent an allergic reaction, asthma, or mast cell activation syndrome. Such prophylactic uses will be of particular value for subjects who are at risk due to an environmental exposure to an allergen or have a genetic predisposition to developing mast cell activation syndrome, severe allergic responses, or asthma.

In another embodiment, the pharmaceutical compositions comprising a tryptase inhibitor, or a derivative thereof, and/or other agents for treating an allergy, asthma, or mast cell activation syndrome, and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

Those of ordinary skill in the art will appreciate which conditions the tryptase inhibitor or a derivative thereof can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

In certain embodiments, multiple therapeutically effective doses of a composition comprising a tryptase inhibitor or a derivative thereof will be administered according to a daily dosing regimen or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, every other week, and so forth. For example, in some embodiments, a composition comprising a tryptase inhibitor will be administered once-weekly, twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., once-weekly, twice-weekly or thrice-weekly administration of a therapeutically effective dose) for one or more weekly cycles until the desired therapeutic response is achieved. The agents can be administered by any acceptable route of administration as noted herein below. The amount administered will depend on the potency of the tryptase inhibitor or the derivative thereof and/or other agents administered, the magnitude of the effect desired, and the route of administration.

A tryptase inhibitor or a derivative thereof (again, preferably provided as part of a pharmaceutical preparation) can be administered alone or in combination with one or more other therapeutic agents, such as other agents for treating an allergy, asthma, or mast cell activation syndrome, including, but not limited to, mast cell stabilizers, including cromolyn sodium and quercetin; antihistamines, including cetirizine, ketotifen, fexofenadine, loratadine, ranitidine, famotidine, levocetirizine, and diphenhydramine; non-steroidal anti-inflammatory drugs, including aspirin, diflunisal, ibuprofen, dexibuprofen, naproxen, fenoprofen, ketoprofen, dexketoprofen, indomethacin, tolmetin, sulindac, etodolac, ketorolac, piroxicam, meloxicam, tenoxicam, droxicam, mefenamic acid, and meclofenamic acid; beta 2-adrenoceptor agonists, including salbutamol (albuterol); corticosteroids, including budesonide, fluticasone, mometasone and ciclesonide; or other medications used to treat a particular condition or disease according to a variety of beta 2-adrenoceptor agonists, including salbutamol (albuterol); corticosteroids, including budesonide, fluticasone, mometasone and ciclesonide; dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

The tryptase inhibitor or a derivative thereof can be administered prior to, concurrent with, or subsequent to other agents. If provided at the same time as other agents, a tryptase inhibitor or a derivative thereof can be provided in the same or in a different composition. Thus, a tryptase inhibitor or a derivative thereof and one or more other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising a tryptase inhibitor or a derivative thereof and a dose of a pharmaceutical composition comprising at least one other agent, such as another drug for treating an allergy, asthma, or mast cell activation syndrome, which in combination comprise a therapeutically effective dose, according to a particular dosing regimen. Similarly, a tryptase inhibitor or a derivative thereof and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

Toxicity can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

Additionally, treatment with a tryptase inhibitor or a derivative thereof may be combined with any other medical treatment for an allergy, asthma, or mast cell activation syndrome, such as, but not limited to, administering mast cell stabilizers, including cromolyn sodium and quercetin; antihistamines, including cetirizine, ketotifen, fexofenadine, loratadine, ranitidine, famotidine, levocetirizine, and diphenhydramine; non-steroidal anti-inflammatory drugs, including aspirin, diflunisal, ibuprofen, dexibuprofen, naproxen, fenoprofen, ketoprofen, dexketoprofen, indomethacin, tolmetin, sulindac, etodolac, ketorolac, piroxicam, meloxicam, tenoxicam, droxicam, mefenamic acid, and meclofenamic acid; beta 2-adrenoceptor agonists, including salbutamol (albuterol); corticosteroids, including budesonide, fluticasone, mometasone and ciclesonide; or any combination thereof.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-18 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A composition comprising a tryptase inhibitor for use in a method of treating an allergy, asthma, or mast cell activation syndrome.
2. The composition of aspect 1, wherein the tryptase inhibitor is Avoralstat.
3. The composition of aspect 1 or 2, further comprising a pharmaceutically acceptable excipient.
4. The composition of any one of aspects 1 to 3, further comprising a pharmaceutically acceptable carrier selected from the group consisting of a cream, emulsion, gel, liposome, nanoparticle, or ointment.
5. The composition of any one of aspects 1 to 4, wherein the allergy is a peanut allergy.
6. The composition of any one of aspects 1 to 5, wherein the tryptase inhibitor inhibits tryptase b2 or tryptase g1 or both tryptase b2 and tryptase g1.
7. A method of treating a subject for an allergy, asthma, or mast cell activation syndrome, the method comprising administering a therapeutically effective amount of a composition comprising a tryptase inhibitor to the subject.
8. The method of aspect 7, wherein the tryptase inhibitor is Avoralstat.
9. The method of aspect 7 or 8, wherein multiple cycles of treatment are administered to the subject.
10. The method of aspect 9, wherein the tryptase inhibitor is administered according to a daily dosing regimen or intermittently.
11. The method of any one of aspects 7 to 10, wherein the tryptase inhibitor is administered orally or intravenously.
12. The method of any one of aspects 7 to 11, wherein the subject is a human.
13. The method of any one of aspects 7 to 12, further comprising administering an antihistamine or non-steroidal anti-inflammatory drug.
14. The method of any one of aspects 7 to 13, wherein the composition further comprises a pharmaceutically acceptable excipient.
15. The method of any one of aspects 7 to 14, wherein the composition further comprises a pharmaceutically acceptable carrier selected from the group consisting of a cream, emulsion, gel, liposome, nanoparticle, or ointment.
16. The method of any one of aspects 7 to 15, wherein the allergy is a peanut allergy.
17. The method of any one of aspects 7 to 16, wherein the tryptase inhibitor inhibits tryptase b2 or tryptase g1 or both tryptase b2 and tryptase g1.
18. A method of inhibiting tryptase b2 or tryptase g1, the method comprising contacting tryptase b2 or tryptase g1 with Avoralstat.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### Drug Repurposing for Treatment of Asthma and Peanut Allergy

Even though the role of tryptases in MCAS is not clearly understood, based on the fact that the abundant secretion of tryptase is strongly correlated with MCAS, inhibiting activity of tryptases is a desired therapeutic strategy to attenuate severe allergic reactions. It is thought that different subtypes of tryptase are responsible for different triggering compounds, including tryptase b2 for asthma and peanut allergy. Since tryptases are part of the S1-peptidase family containing other proteases, whose inhibitors have been extensively investigated, we strategized to utilize a protein-structural similarity-based drug repurposing.

### Results

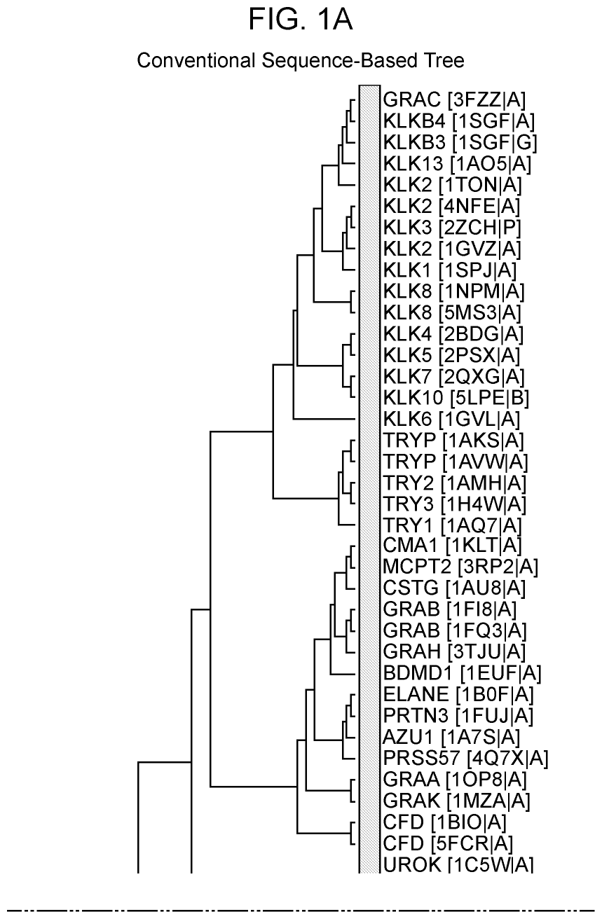

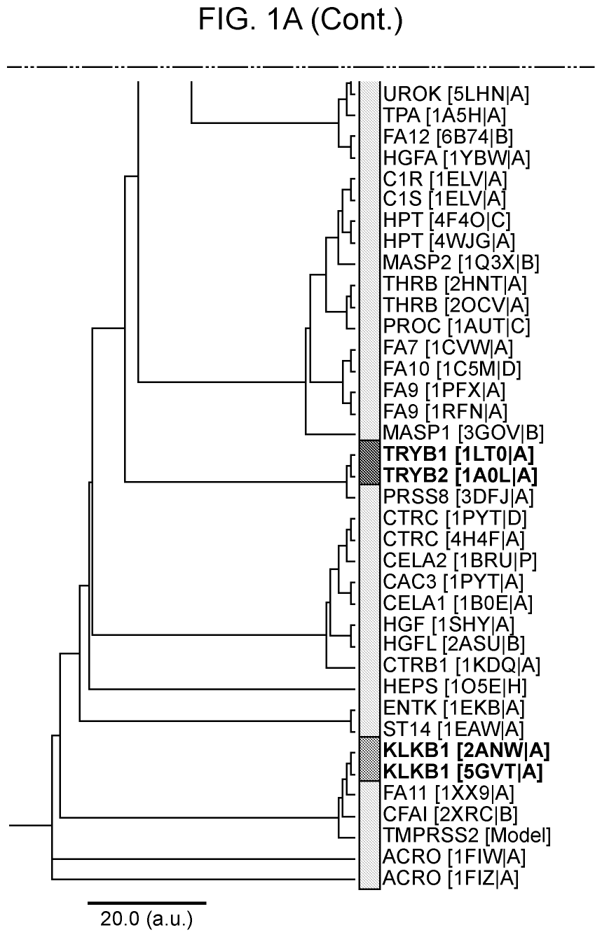

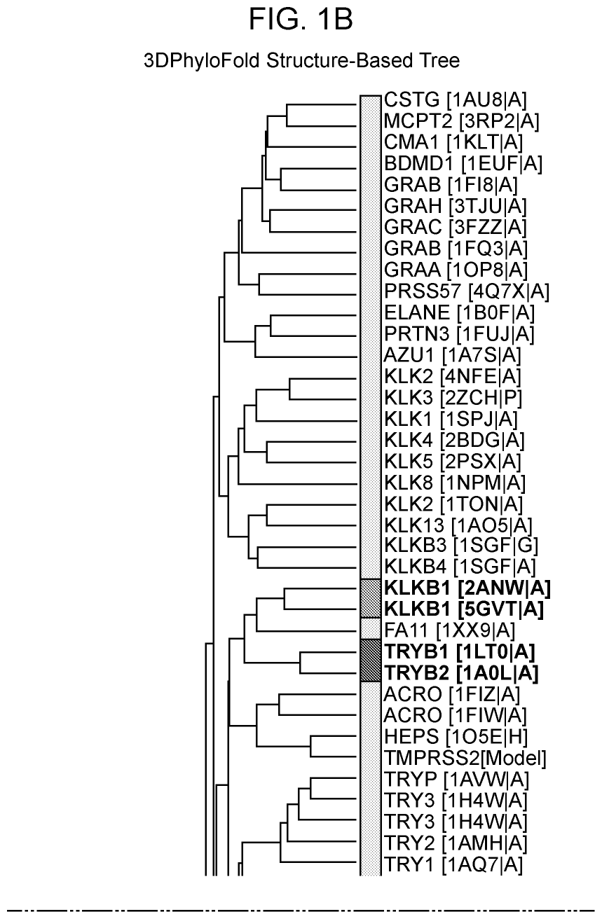

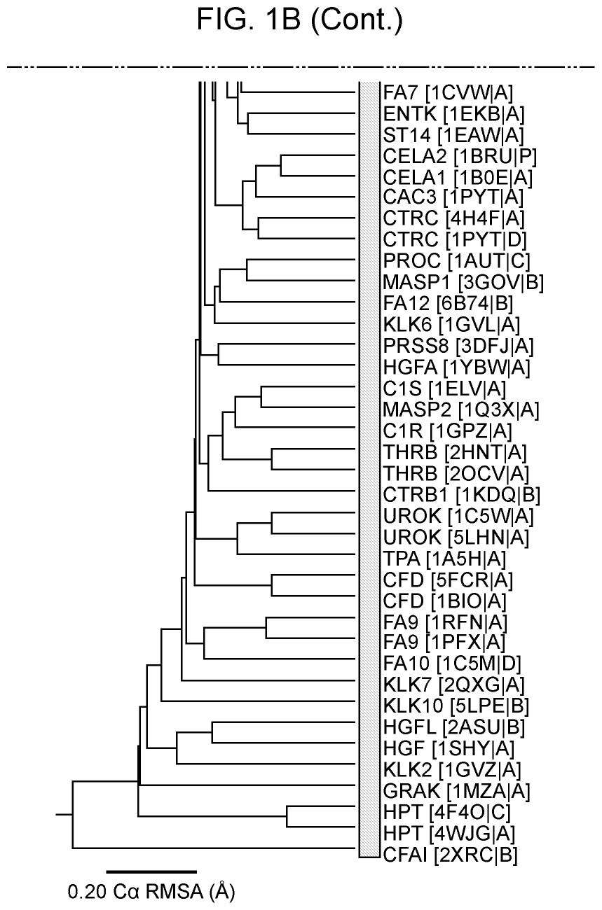

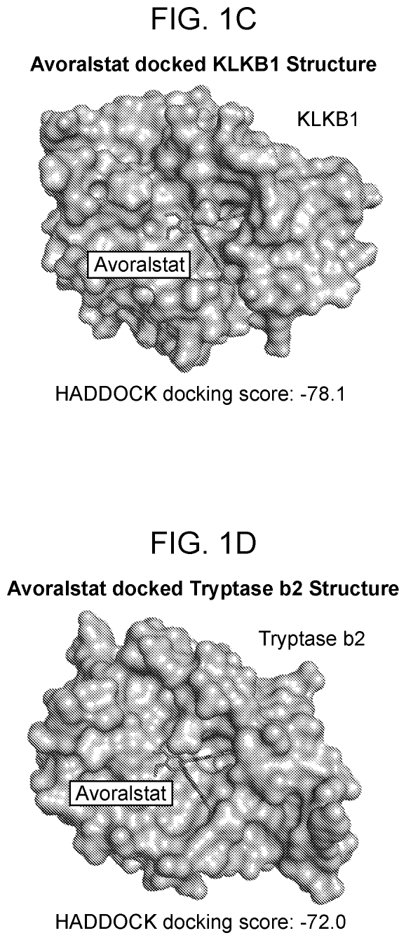

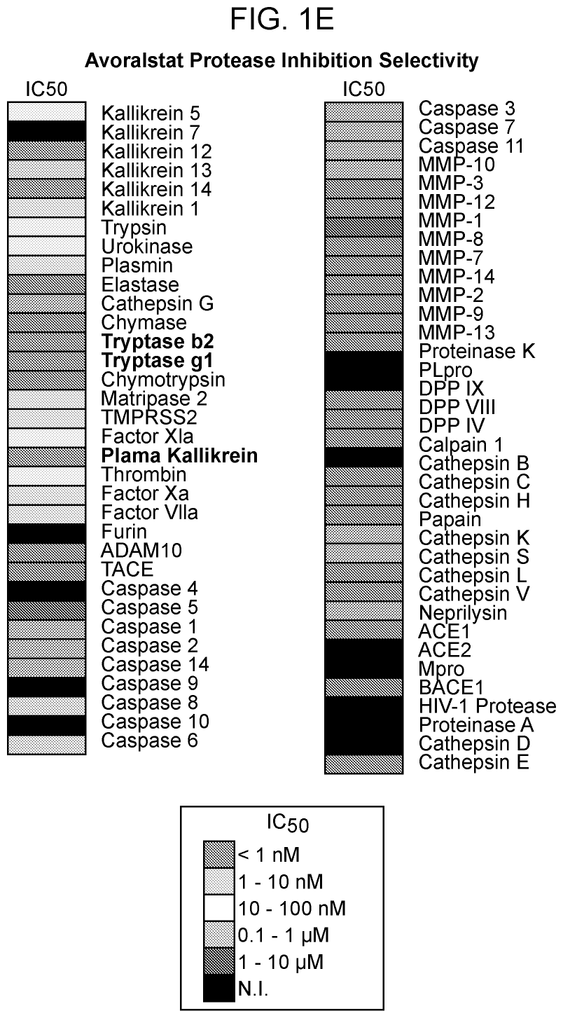

In silico drug repositioning by 3DPhyloFold identified Avoralstat as a primary candidate for Tryptase inhibition. We created a computational/hypothesis-driven drug repurposing method called 3DPhyloFold that identifies structurally similar proteins to rationally select candidate inhibitors. 3DPhyloFold determined the 3D relationship of Tryptases to other S1-peptidase structures. Using structural quality metrics (see Methods), 73 S1-peptidases, Tryptase-b1 and -b2 were aligned by conventional sequence phylogenetic analysis. Tryptases and only Prostasin (PRSS8) form an exclusive cluster, and other proteases were distantly related to Tryptases (FIG. 1A). In 3DPhyloFold, pairwise structural comparisons of the representative tertiary structures were used to calculate a structural dissimilarity matrix (SDM) based on the root mean square deviation between protein alpha-carbons (Cα RMSD). A structure-based phylogenetic tree was then generated from the SDM (FIG. 1B). Clustering of the structure-based tree was distinct to that of the sequence-based tree. Protein close in the primary sequence analysis, PRSS8, were much farther away in the 3DPhyloFold structure-tree. Although distant in the sequence-based tree, the Plasma Kallikrein (KLKB1) clade moved much closer to Tryptases in the structure-based tree (FIG. 1B). This suggested that, while divergent in sequence, Tryptases adopts a three-dimensional fold closer to KLKB1. Thus, we prioritized KLKB1 inhibitors as repurposing candidates for Tryptase inhibition.

We have identified Avoralstat as one of the inhibitors for KLKB1, which is an oral drug that is clinically proven to be safe in its phase Ill clinical trials for hereditary angioedema. We have performed an in silico docking calculation for Avoralstat against KLKB1 and Tryptase-b2 using online HADDOCK docking tool (FIGS. 1C-1D). The avoralstat's docking score to each protease was comparable (−78.1 for KLKB1 and −72.0 for Tryptase-b2; lower the score, stronger the binding) suggesting that Avoralstat may inhibit Tryptase-b2 as well as it inhibits it original target KLKB1.

Avoralstat can inhibit Tryptase-b2 and -g1 as well as it inhibits KLKB1 in vitro. Avoralstat was assessed for inhibition against a panel of recombinant proteases including 2 Tryptases (Tryptase-b2 and -g1) and KLKB1 by commercial services from Reaction Biology Corp. The Reaction Biology Corp profile tested in a 10-dose IC₅₀, with a dose range from 38 pM to 10 μM, against 70 proteases in FIG. 1E. Avoralstat inhibited KLKB1 with an IC₅₀ of 0.13 nM, while it inhibited Tryptase-b2 with an IC₅₀ of 0.99 nM and Tryptase-g1 with an IC₅₀ of 0.93 nM (FIG. 1E). Only two other proteases, Kallikrein-12 and Kallikrein-14, showed sub-nanomolar inhibition. This in vitro biochemical data suggests that Avoralstat has high potential to be repurposed to inhibit Tryptases and may serve as a therapeutic to attenuate the symptoms of Mast Cell Activation Syndrome.

### Methods

Database search and sequence alignment. We first searched the UniProt database for reviewed entries denoted as transmembrane serine proteases (containing an S1-peptidase domain). This initial search yielded 9 manually curated sequences. A seed multiple sequence alignment (MSA) of S1-peptidase domains was then constructed using MAFFT v7 (alignment strategy: FFT-NS-1) (26). Using HMMER-3.1 and the seed alignment, we produced an HMM profile and used it to broaden the search against the UniProt database (search restricted to reviewed sequences) (27). This search yielded a total of 828 S1-peptidase sequences. We discarded fragmented sequences (<200 amino acids) that appeared too short to truly represent the S1-peptidase fold and redundant proteins were further filtered using CD-HIT v4 (100% threshold) (28). This resulted in a pool of 742 proteins that were aligned using MAFFT v7 (alignment strategy FFT-NS-2) (26). Sequences producing many gaps in the alignment were removed using MaxAlign, resulting in 600 S1-peptidase sequences (29).

Phylogenetic tree reconstruction. We used the IQ-TREE-1.6.2 algorithm to generate a maximum likelihood tree of the 600 S1-peptidase sequences (30). The IQ-TREE model finder tool was used to determine the best substitution model to fit the data. The Whelan & Goldman (WAG) substitution model was determined to be the best fit to the data. Bootstrap analysis was performed using the ‘ultra-fast’ method in IQ-TREE-1.6.2 with 1,000 replicas.

Structure-based phylogenetic analysis. There are over 2,000 structures of S1-peptidase domains represented in the PDB. We therefore searched the Pfam database for structures of mammalian peptidases and selected 74 representative structures (representing the wild-type protein) with an atomic resolution 3.2 Å or better. One structure per unique protein, fitting the above criteria, was selected. Structures (with reflection data deposited in the PDB) were evaluated by their reported global validation metrics in PDB-REDO. Re-refined structural models were used for further analysis. Structures were superimposed using PyMOL to calculate the pairwise root mean square deviation (RMSD) between protein alpha carbon atoms (Cα). A structural dissimilarity matrix (SDM) was constructed using the Cα RMSD values in order to generate a phylogenetic tree as previously described. To expedite the pairwise alignment process, we developed a Python-based script (named 3DPhyloFold) to perform the pairwise alignment of protein structures and generate an SDM. The phylogenetic tree was constructed using the UPGMA (Unweighted Pair Group Method with Arithmetic Mean) method in MEGAX software. For comparison, the sequences from the corresponding structures were also analyzed by sequence-based phylogeny. The 75 S1-peptidase sequences were aligned with MAFFT v7 and analyzed in IQ-TREE-1.6.2. The Jones-Taylor-Thornton (JTT) substitution model was determined to be the best fit to the data. Bootstrap analysis was performed in IQ-TREE-1.6.2 (1,000 replicas).

In silico docking of Avoralstat to KLKB1 and Tryptase-b2. The HADDOCK 2.4 online docking tool was used to generate KLKB1/Avoralstat and Tryptase-b2/Avoralstat complex structure model. The KLKB1 structure (PDB 601S) and Tryptase-b2 structure (PDB 1AOL) deposited in protein data bank were used for docking. The potential interaction surface between Avoralstat and each S1-peptidase was defined by the conserved active site of S1-peptidase family. The HADDOCK scores represent the average score of the best cluster.

Protease activity array. Avoralstat was assessed for inhibition against TMPRSS2 and a panel of recombinant proteases by commercial services from Reaction Biology Corp. The Reaction Biology Corp profile tested in a 10-dose IC₅₀, in triplicate, with a 3-fold serial dilution starting at 10 μM against 70 proteases in FIGURE E. Compounds exhibit no fluorescent background that could interfere with the assay. The protease activities were monitored as a time-course measurement of the increase in fluorescence signal from fluorescently labeled peptide substrate, and initial linear portion of slope (signal/min) was analyzed.

1-6. (canceled)

7. A method of treating a subject for an allergy, asthma, or mast cell activation syndrome, the method comprising administering a therapeutically effective amount of a composition comprising a tryptase inhibitor to the subject.

8. The method of claim 7, wherein the tryptase inhibitor is Avoralstat.

9. The method of claim 7, wherein multiple cycles of treatment are administered to the subject.

10. The method of claim 9, wherein the tryptase inhibitor is administered according to a daily dosing regimen or intermittently.

11. The method of claim 7, wherein the tryptase inhibitor is administered orally or intravenously.

12. The method of claim 7, wherein the subject is a human.

13. The method of claim 7, further comprising administering an antihistamine, a non-steroidal anti-inflammatory drug, a beta 2-adrenoceptor agonist, or a corticosteroid.

14. The method of claim 7, wherein the composition further comprises a pharmaceutically acceptable excipient.

15. The method of claim 7, wherein the composition further comprises a pharmaceutically acceptable carrier selected from the group consisting of a cream, emulsion, gel, liposome, nanoparticle, or ointment.

16. The method of claim 7, wherein the allergy is a peanut allergy.

17. The method of claim 7, wherein the tryptase inhibitor inhibits tryptase b2 or tryptase g1 or both tryptase b2 and tryptase g1.

18. A method of inhibiting tryptase b2 or tryptase g1, the method comprising contacting tryptase b2 or tryptase g1 with Avoralstat.
